(19) United States
(12) Patent Application Publication
(10) Pub. No.: US 2007/0213742 A1
(43) Pub. Date: Sep. 13, 2007

(54) Surgical hair trimmer

(76) Inventor: Mark J. Callahan, Wilbraham, MA (US)

Correspondence Address: CARTER, DELUCA, FARRELL & SCHMIDT, LLP, 445 BROAD HOLLOW ROAD, SUITE 225, MELVILLE, NY, 11704, US

(21) Appl. No.: 11/716,077

(22) Filed: Mar. 8, 2007

Related U.S. Application Data
(60) Provisional application No. 60/780,335, filed on Mar. 8, 2006.

(51) Int. Cl. A61B 17/50

(52) U.S. Cl. 606/133

(57) ABSTRACT

A surgical hair trimmer is provided including a handle assembly including a power source, a drive motor connected to the power source and having a drive shaft extending therefrom, and a release mechanism configured and adapted for single handed operation; and a blade assembly including a pair of blades wherein at least one of the pair of blades is movable with respect to the other of the pair of blades, an interconnect configured and adapted to operatively couple the drive shaft to the movable blade when the blade assembly is connected to the handle assembly, and engagement structure configured and adapted to selectively engage the release mechanism of the handle assembly. In use, actuation of the release mechanism, when the blade assembly is connected to the handle assembly, jettisons the blade assembly from the handle assembly.

### REFERENCE TO RELATED APPLICATION

The present application claims the benefit of and priority to U.S. Provisional Application 60/780,335, filed on Mar. 8, 2006, the entire content of which is incorporated herein by reference.

### BACKGROUND

1. Technical Field

The present disclosure relates generally to medical devices and, more particularly, to surgical hair trimmers.

2. Discussion of Related Art

Technological advances in medicine have allowed for hair removal to be achieved by a variety of means, including and not limited to razors, depilatory agents, and electric hair trimmers.

A drawback to razor preparation of irregular or bony areas is that such preparation often results in either inadequate hair removal and/or injury, such as, irritation, inflammation, nicks and scratches. Such injury to the skin, as a result of razor preparation, may result in an increase in the rate of infection.

A drawback to the use of depilatory agents is that use of depilatory agents may result in irritation and/or an allergic reaction (i.e., rash) in the patient. However, the use of depilatory agents often results in fewer disruptions of skin integrity as compared to razor preparation.

It has been found that either hair trimmers or depilatory agents have less potential of disrupting skin integrity. Since hair trimmers result in less irritation to the skin as compared to the use of depilatory agents and since hair trimmers result in fewer skin disruptions as compared to razors, the use of clippers to prepare the skin of a patient prior to a surgical procedure has emerged as the preferred method of skin preparation.

Therefore, a need exists for improved and more efficient surgical hair trimmers and the like.

The improved surgical hair trimmers should reduce the preparation time required for a patient thus reducing the time the patient spends in the operating room and thus the total cost for the use of the operating room.

The improved surgical hair trimmers should reduce incidents of clogging.

The improved surgical hair trimmers should reduce the amount of clinician contact with sharps and the like, such as, the blade thereof.

The improved surgical hair trimmers should reduce the amount of clinician contact with the hair of the patient either prior to or after the clipping procedure.

### SUMMARY

The present disclosure relates surgical hair trimmers. According to an aspect of the present disclosure, a surgical hair trimmer is provided and comprises a handle assembly including a handle housing configured and dimensioned to house a power supply and a drive motor, wherein the drive motor includes a drive shaft extending through a distal end of the handle housing; and a release mechanism associated with the handle housing. The release mechanism includes at least one tang having a proximal end extending into the handle housing and a distal end extending out of the handle housing and is configured to engage a blade assembly. The release mechanism further includes at least one release button operatively associated with a respective tang.

The surgical hair trimmer further includes a blade assembly selectively connectable to the handle assembly. The blade assembly includes a blade housing configured and dimensioned to house a pair of trimmer blades; an interconnect operatively connected to one of the pair of trimmer blades to impart movement to the one trimmer blade, the interconnect being configured and adapted to selectively operatively engage the drive shaft of the drive motor when the blade assembly is connected to the handle assembly; and an engagement structure formed in blade housing for selectively engaging the distal end of each tang of the release mechanism.

In use, when the blade assembly is connected to the handle assembly the distal end of each tang of the release mechanism of the handle assembly engages with a corresponding engagement structure of the blade housing of the blade assembly. Moreover, upon actuation of the at least one release button of the release mechanism jettisons the blade assembly from the handle assembly.

The surgical hair trimmer may include a pair of release buttons provided on side surfaces of the handle housing for operative engagement with at least a pair of tangs. The surgical hair trimmer may include a single release button provided on a rear end of the handle housing for operative engagement with at least one tang. Each of the release buttons may be over-molded.

It is contemplated that the distal end of at least one tang extends in an outward direction.

The surgical hair trimmer may include at least one visual indicator supported on the handle housing. The surgical hair trimmer may further include a shroud extending from a distal end of the handle housing.

The drive shaft of the drive motor may extend through a distal wall of the handle housing. The surgical hair trimmer may include a sealing member interposed between the drive shaft and the distal wall of the handle housing.

The surgical hair trimmer may further include an identification tag associated with the handle assembly for recognition with a corresponding identification tag of a charging base. The surgical hair trimmer may be configured for use with an anti-theft system, wherein the anti-theft system includes an electrical outlet connector configured and adapted for electrical connection to an electrical outlet and defining a proprietary configuration of electrical contact receptacles; and a power cord including a power cord connector electrically connected to an end thereof. The power cord connector may include electrical contacts complementing the electrical contact receptacles of the electrical outlet connector.

According to another aspect of the present disclosure, a surgical hair trimmer is provided which comprises a handle assembly and a blade assembly. The handle assembly includes a power source, a drive motor connected to the power source and having a drive shaft extending therefrom, and a release mechanism configured and adapted for single handed operation; and a blade assembly. The blade assembly includes a pair of blades wherein at least one of the pair of blades is movable with respect to the other of the pair of blades, an interconnect configured and adapted to operatively couple the drive shaft to the movable blade when the blade assembly is connected to the handle assembly, and engagement structure configured and adapted to selectively engage the release mechanism of the handle assembly.

In use, actuation of the release mechanism, when the blade assembly is connected to the handle assembly, jettisons the blade assembly from the handle assembly.

The release mechanism may include at least one tang having a proximal end extending into a handle housing of the handle assembly and a distal end extending out of the handle housing. The distal end of each tang may be configured to engage the blade assembly; and at least one release button operatively associated with a respective tang.

The engagement structure may be formed in a blade housing of the blade assembly for selectively engaging the distal end of each tang of the release mechanism.

A pair of release buttons may be are provided on side surfaces of the handle housing for operative engagement with at least a pair of tangs. A single release button may be provided on a rear end of the handle housing for operative engagement with at least one tang. Each release button may be over-molded.

The handle assembly may include at least one visual indicator. A distal end of a handle housing of the handle assembly may include a shroud extending therefrom.

The drive shaft of the drive motor may extend through a distal wall of a handle housing of the housing assembly. A sealing member may be interposed between the drive shaft and the distal wall of the handle housing.

The surgical hair trimmer may include an identification tag associated with the handle assembly for recognition with a corresponding identification tag of a charging base.

The surgical hair trimmer may be configured for use with an anti-theft system, wherein the anti-theft system which includes an electrical outlet connector configured and adapted for electrical connection to an electrical outlet and defining a proprietary configuration of electrical contact receptacles; and a power cord including a power cord connector electrically connected to an end thereof, wherein the power cord connector includes electrical contacts complementing the electrical contact receptacles of the electrical outlet connector.

### DETAILED DESCRIPTION OF THE DRAWINGS

Various embodiments of the presently disclosed surgical hair trimmers are disclosed herein with reference to the drawings, wherein:

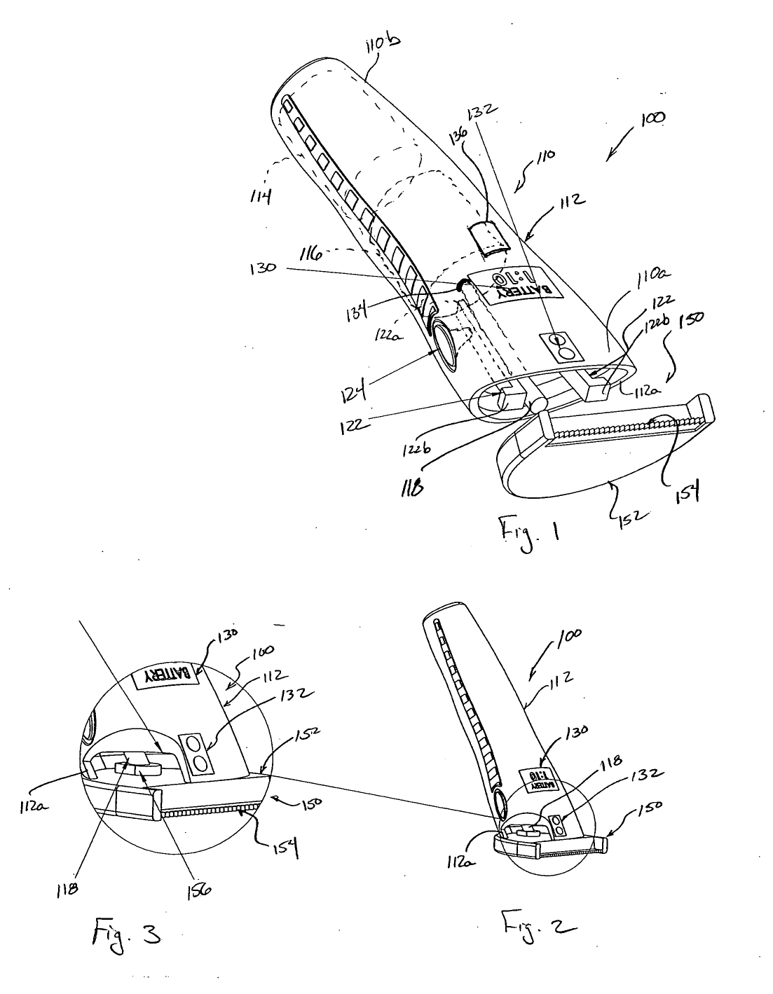

FIG. 1 is a schematic, perspective view of a surgical hair trimmer according to an embodiment of the present disclosure, illustrating a blade assembly separated from a handle assembly;

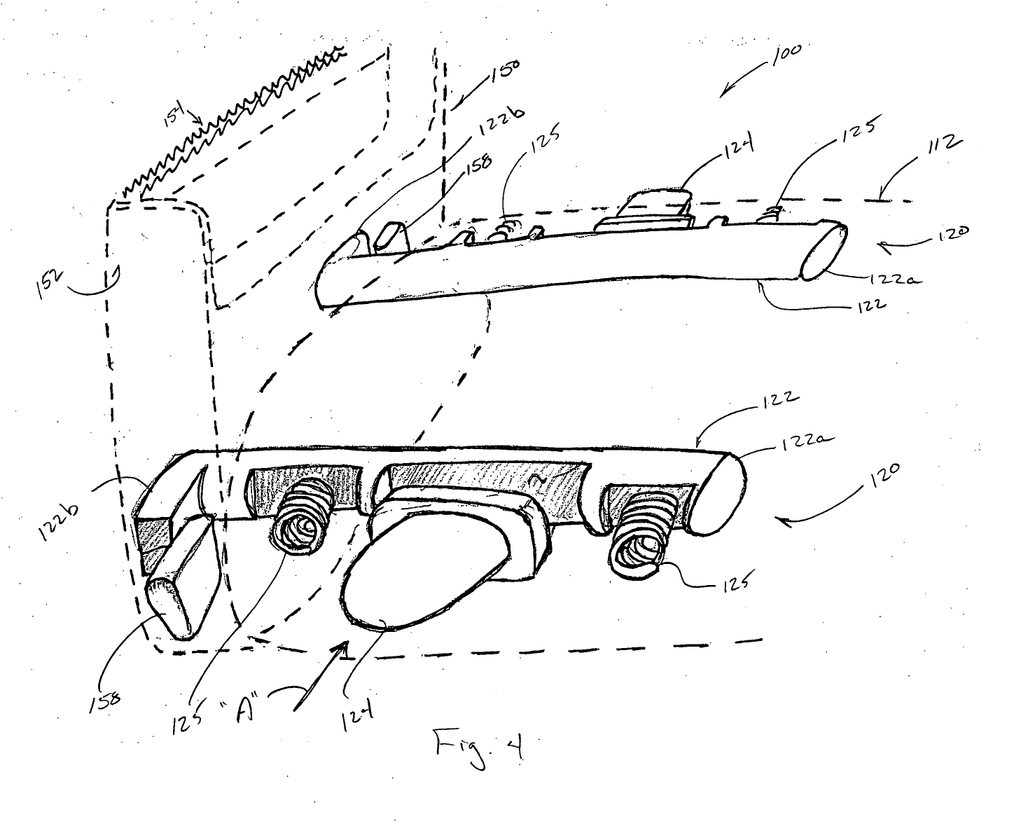

FIG. 2 is a schematic, perspective view, partially broken away, of the surgical hair trimmer of FIG. 1, illustrating the blade assembly operatively connected to the handle assembly;

FIG. 3 is an enlarged view of the indicated area of detail of FIG. 2;

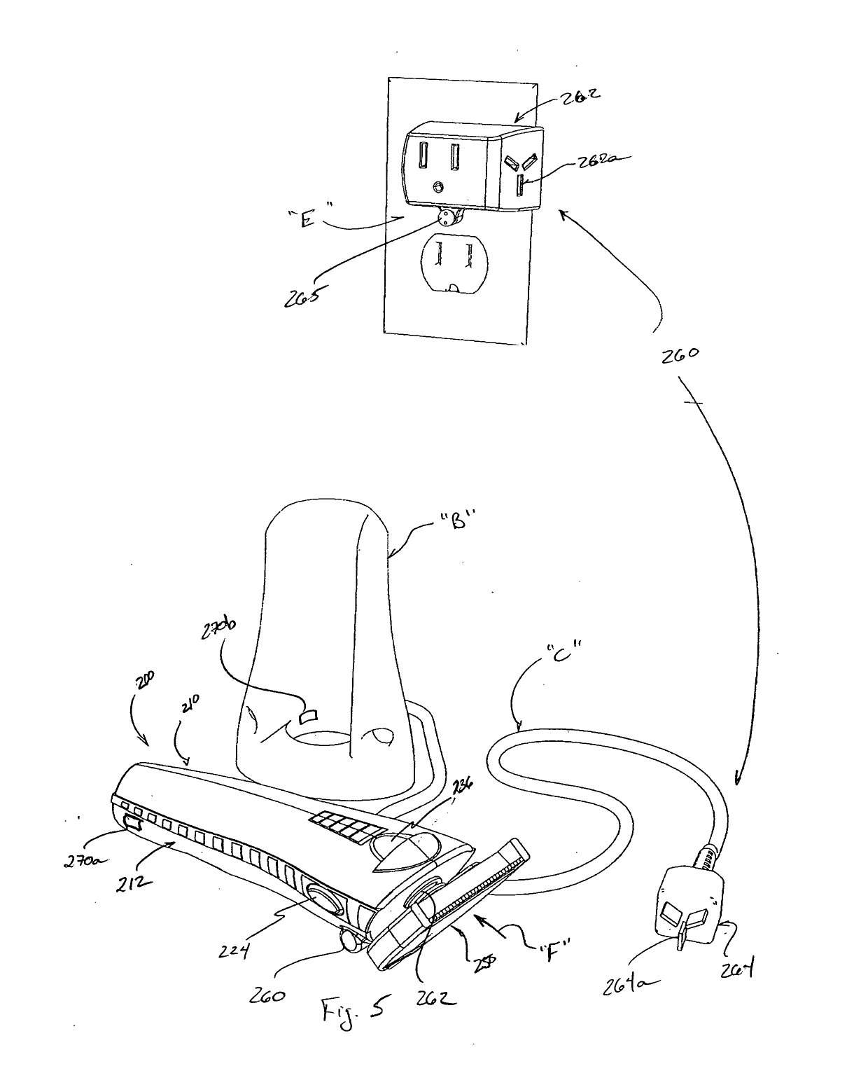

FIG. 4 is a schematic, perspective view of the surgical hair trimmer of FIGS. 1-3, illustrating the inner components of a release mechanism thereof;

FIG. 5 is an illustration of an exemplary surgical hair trimmer and anti-theft system, according to an embodiment of the present disclosure;

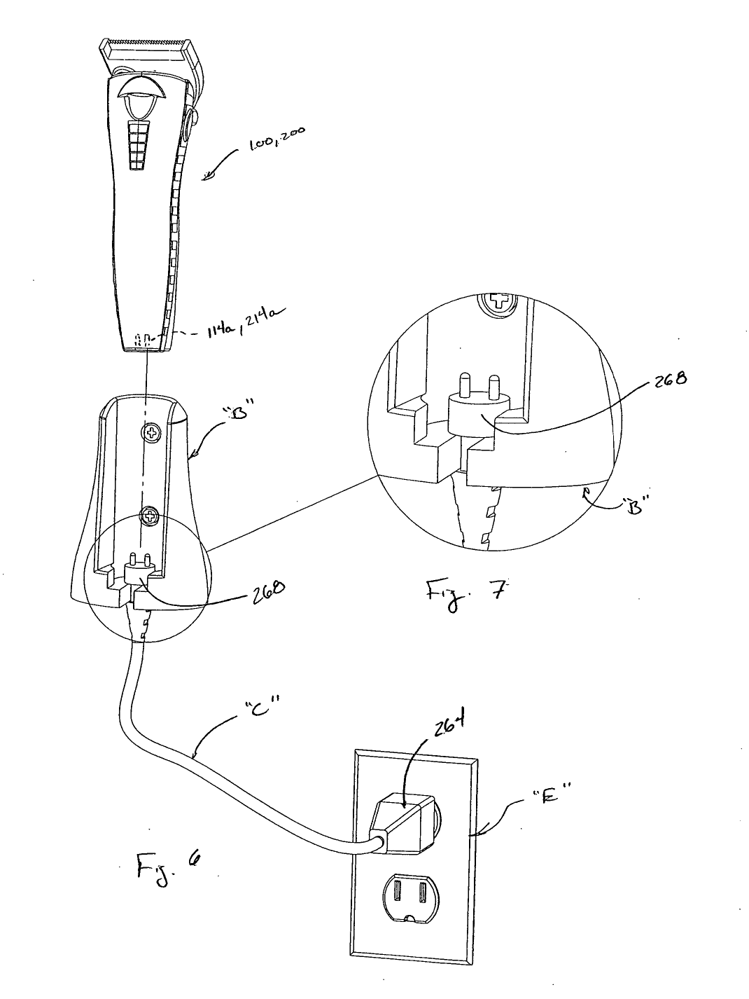

FIG. 6 is an illustration of a surgical hair trimmer system including a selectively usable recharge cord;

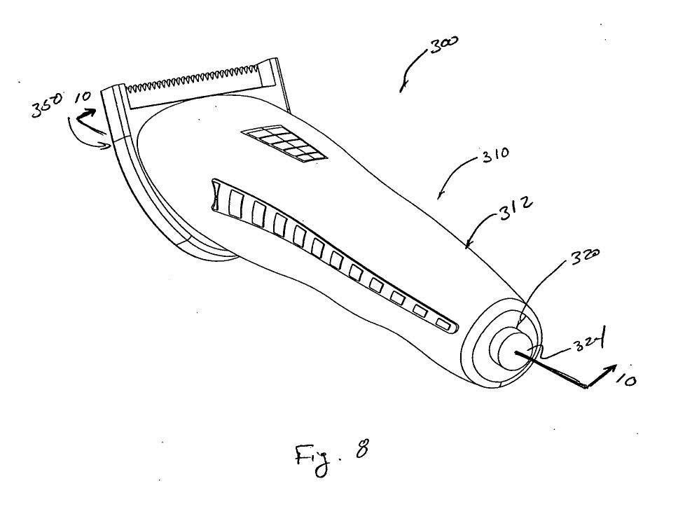

FIG. 7 is an enlarged view of the indicated area of detail of FIG. 6;

FIG. 8 is a rear, perspective view of a surgical hair trimmer according to another embodiment of the present disclosure;

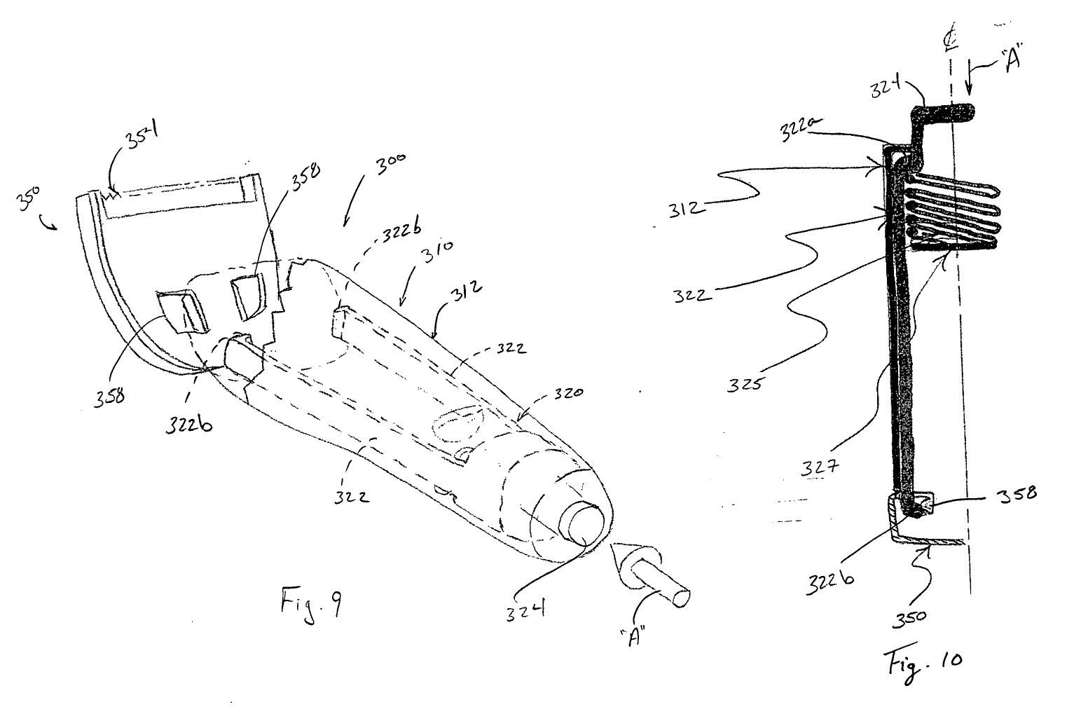

FIG. 9 is a rear, perspective view of the surgical hair trimmer of FIG. 8, illustrating the inner components of a release mechanism thereof; and

FIG. 10 is a cross-sectional view of the surgical hair trimmer of FIGS. 8 and 9, as taken through 10-10 of FIG. 8.

### DETAILED DESCRIPTION OF EMBODIMENTS

The surgical hair trimmers of the present disclosure provide the operator, e.g., health care professional, with an increased ability to remove hair from a target surgical site prior to performing a surgical procedure. Moreover, the surgical hair trimmers of the present disclosure provide for a safe, reliable and efficient method of removing hair from the target surgical site while minimizing the incidents of skin irritation and/or injury.

In the following description, as is traditional, the term “proximal” refers to the portion of the apparatus or device which is closest to the operator while the term “distal” refers to the portion of the apparatus or device which is remote from the operator.

According to an embodiment of the present disclosure, as seen in FIGS. 1-3, a surgical hair trimmer is generally designated as 100. Surgical hair trimmer 100 includes a handle assembly 110 and a selectively removable blade assembly 150 operatively connectable to handle assembly 110.

As seen in FIGS. 1 and 2, handle assembly 110 includes a handle housing 112 having an ergonomic outer profile substantially similar to the outer profile of the any of the outer or fluid housings disclosed in U.S. application Ser. No. 11/200,460, filed on Aug. 8, 2005, the entire content of which is incorporated herein by reference. Handle housing 112 may be made of durable, impact-resistant molded polymeric or plastic material, as is known in the art.

Handle assembly 110 may include a rigid inner casing and a soft material, over-mold giving handle assembly 110 a soft feel. It is contemplated that the over-mold may incorporate bumps, ridges, slots, grooves, dimples, knurling, etc, formed therein or thereon which improves the gripping ability of handle assembly 110. It is contemplated that the handle assembly 110 may have a substantially ovular transverse cross-sectional profile.

Handle assembly 110 includes a drive or distal end 110a, and a recharge or proximal end 110b located substantially opposite drive end 110a. Handle assembly 110 includes a power source 114 (shown in phantom in FIG. 1) housed or retained within handle housing 112. Power source 114 may include, and is not limited to, one or more rechargeable batteries, disposable batteries, or an electric transformer connectable to a power cord which is connectable to an electrical outlet. In the present embodiment, terminals 114a (see FIG. 5) for engaging a power cord or a charging base are located at recharge end 110b of handle assembly 110.

With continued reference to FIG. 1, handle assembly 110 further includes a drive member 116 (shown in phantom), in the form of an electric motor or the like, housed or retained within handle housing 112 and operatively connected to power source 114 in a manner known in the art. Drive member 116 includes a drive shaft 118 extending therefrom and toward drive end 110a of handle assembly 110. Drive shaft 118 may include an eccentric cam (not shown), supported thereon, as known by one having skill in the art. The arrangement and operation of power source 114, drive member 116 and the eccentric cam of drive shaft 118 are similar to components which are well known in the art.

As seen in FIGS. 1-4, handle assembly 110 further includes a blade assembly attachment and release mechanism 120 supported on or in handle housing 112. Release mechanism 120 includes a pair of tangs 122 supported in handle housing 112 proximate drive end 110a of handle assembly 110. Each tang 112 may be pivotally or slidably supported within handle housing 112. Each tang 122 includes a proximal end 122a extending into handle housing 112, and a distal end 122b extending out of a distal end of handle housing 112. Distal end 122b of each tang 122 is out-turned to define a first engagement structure.

Release mechanism 120 further includes at least one push button 124 or the like supported on opposite sides of handle housing 112. Each push button 124 is in operative engagement with at least a respective tang 122, wherein actuation of a push button 124 results in corresponding actuation or movement of a respective tang 122. In operation, as will be discussed in greater detail below, as each push button 124 is pressed inward (in the direction of arrow “A”), a corresponding tang 122 is moved in a first direction, and as each push button 124 is released to move outward (in a direction opposite to arrow “A”), the corresponding tang 122 is moved in a second direction, opposite the first direction. It is contemplated that each push button 124 may be biased, by a suitable biasing member(s) 125, in the form of a compression or tension spring, to maintain the push button 124 in a substantially un-actuated condition.

Release mechanism 120 is configured and adapted to enable the clinician to attach and remove blade assembly 150 to/from handle assembly 110 with a single hand. In particular, as will be discussed in detail below, the clinician may hold handle assembly 110 in a single hand, actuate release mechanism 120 with the same hand, and attach/detach blade assembly 150 to/from handle assembly 110 with the same hand. In this manner, contact of the clinicians other hand, with the blade assembly 150 or the like is eliminated, thereby reducing instances of contact or injury with the sharp of the blade assembly 150. In particular, the clinician may attach a new blade assembly to handle assembly using only a singe hand and may detach the blade assembly from the handle assembly using only a single hand.

With continued reference to FIGS. 1-3, handle housing 112 includes a shroud 112a extending in a distal direction therefrom. In use, shroud 112a extends an amount or distance sufficient to contact or engage a surface of a housing of blade assembly 150. In this manner, debris, such as, hair clippings or the like, is substantially prevented from contaminating release mechanism 120 and/or drive shaft 118.

As seen in FIGS. 1-3, handle assembly 110 may include a visual indicator 130 supported on handle housing 112. Visual indicator 130 may be any suitable display known in the art to provide an indication of an event. The event may be related to the procedure or the operation of the surgical hair trimmer 100, such as, for example, battery life, blade assembly life, etc. Visual indicator 130 may be a liquid crystal display (LCD), a plasma display, one or more light emitting diodes (LEDs), a luminescent display, a multi-color display, a digital display, an analog display, a passive display, an active display, a so called “twisted nematic” display, a so called “super twisted nematic” display, a “dual scan” display, a reflective display, a backlit display, an alpha numeric display, a monochrome display, a so called “Low Temperature Polysilicon Thin Film Transistor” (LPTS TFT) display, or any other suitable display that indicates a parameter, information or graphics related to the procedure or surgical hair trimmer 100.

In one embodiment, visual indicator 130 is a liquid crystal display which may be a black & white or color display that displays one or more operating parameters of surgical hair trimmer 100 to the clinician. In one embodiment, the operating parameter displayed may be the life of the battery, the life of the blade assembly, or any other parameter of the procedure. The visual indicator 130 may display text, graphics or a combination thereof.

In addition to visual indicator 130, handle assembly 110 may incorporate a tactile feedback mechanism (not shown) associated therewith. In this manner, during operation of surgical hair trimmer 100, in addition to or in lieu of visual indicator 130, the clinician is advised of the conditions or parameters of surgical hair trimmer 100 as a result of a vibratory response generated by the tactile feedback mechanism. It is contemplated that any suitable tactile feedback mechanism, known in the art, may be incorporated into handle assembly 110.

The tactile feedback mechanism may be configured to activate when the power level of the surgical hair trimmer (e.g., the battery charge) drops below a predetermined threshold level. It is envisioned that the tactile feedback mechanism may cause the surgical hair trimmer to vibrate at different intensities depending on the particular power level remaining.

As seen in FIGS. 1-3, handle assembly 110 may further include second visual indicator 132 providing the clinician with a visual indication of when blade assembly 150 is properly connected to handle assembly 110 and/or when no blade assembly 150 is connected to handle assembly 110. It is envisioned that Visual indicator 130 may be configured and adapted to accommodate the function of visual indicator 132.

As seen in FIG. 1, handle assembly 110 may include a seal 134, in the form of an O-ring, gasket, bushing or other structure suitable for the intended function, disposed about drive shaft 118 and against a surface of handle housing 112 to thereby seal handle housing 112 against any contaminants, e.g., water, saline, hair clippings, etc., into a chamber of handle housing 112 containing the power source 114 and the drive member 116.

As seen in FIG. 1, handle assembly 110 includes an activation button or slide 136 supported on handle housing 112. Activation button or slide 136 functions to activate and de-activate drive member 116, as desired or needed, by the clinician. Activation button or slide 136 may be supported on handle housing 112 and connected to each of power source 114 and drive member 116 according to any suitable method known in the art.

With continued reference to FIGS. 1-4, a detailed discussion of blade assembly 150 is provided. Blade assembly 150 includes a blade housing 152 configured and adapted to operatively support a pair of hair trimmer blades 154 therein. As is conventional, one of the pair of hair trimmer blades is stationary, while the other of the hair trimmer blades 154 is slidably moveable with respect to the stationary blade.

As seen in FIGS. 2 and 3, blade assembly 150 includes a drive shaft interconnect 156 extending from blade housing 152 and being configured and adapted to operatively engage with drive shaft 118 if handle assembly 110, in particular, with the eccentric cam of drive shaft 118. Interconnect 156 is also operatively connected to the movable or slidable blade of the pair of blades 154, such that, upon rotation of drive shaft 118, reciprocating linear motion is imparted to the movable or slidable blade of the pair of blades 154 relative to the fixed or stationary blade of the pair of blades 154.

As seen in FIG. 4, blade assembly 150 includes an engagement structure 158 formed in a surface of blade housing 152. Engagement structure 158 is configured and adapted to selectively engage with distal end 122b of each tang 122 of release mechanism 120 of handle assembly 110. Engagement structure 158 is configured and adapted such that a secure engagement between handle assembly 110 and blade assembly 150 is achieved when blade assembly 150 is coupled to handle assembly 110. Additionally, engagement structure 158 is configured and adapted such that upon actuation of the release buttons 124, blade assembly 150 is automatically separated from handle assembly 110.

It is envisioned that release mechanism 120 is spring loaded such that when the release buttons 124 are actuated, a spring force or the like acts on blade assembly 150 to push or jettison blade assembly 150 off of or away from handle assembly 110, preferably into a sharps container or the like.

Turning now to FIG. 5, a surgical hair trimmer according to an alternate embodiment of the present disclosure is generally designated as 200. Surgical hair trimmer 200 is substantially identical to surgical hair trimmer 100 and thus will only be discussed further herein to the extent necessary to identify differences in construction and operation.

As seen in FIG. 5, handle assembly 210 includes push buttons 224 which are over-molded with a resilient polymeric material so as to reduce and/or prevent contaminants from entering handle housing 212 therefrom. It is further contemplated that activation switch 236 may be over-molded in a manner similar to push buttons 224.

It is contemplated that the over-molding may extend over any buttons, switches or the like which are provided on handle assembly 110. In so doing, the over-mold seals said buttons, switches and the like from contamination or the like.

With continued reference to FIG. 5, surgical hair trimmer 200 may be utilized with an anti-theft system 260. Anti-theft system 260 includes an electrical outlet connector 262 configured and adapted for electrical connection with a standard electrical outlet. Outlet connector 262 defines a proprietary or unique configuration for electrical contact receptacles 262a. Outlet connector 262 may also be secured to an electrical outlet “E” via a locking screw 265 of the like which is not readily removable by an individual without a special tool.

Anti-theft system 260 further includes a power cord connector 264 electrically supported on an end of a power cord “C”. Power cord connector 264 includes a set of electrical contacts 264a corresponding to or complimentary with the proprietary or unique electrical contact receptacles 262a of outlet connector 262. In this manner, power cord “C” may only be electrically connected to electrical outlet “E” though the proprietary connection of power cord connector 264 and outlet connector 262.

As seen in FIGS. 6 and 7, an opposite end of power cord “C” includes a connector 268 having a proprietary configuration which may be supported in or connected to a cradle or base “B” and may be configured and adapted to electrically engage with terminals 114a, 214a (see FIG. 6) of handle assembly 100, 200. Terminals 114a, 214a of handle assembly 100, 200 may be shaped to mate solely with connector 268. In this manner, when surgical hair trimmer 100, 200 is placed or docked in base “B” terminals 114a, 214a thereof electrically engage connector 268 of power cord “C”. It is envisioned that connector 268 of power cord “C” may be directly connected to surgical hair trimmer 100, 200, and bypass base “B” all together.

Turning back to FIG. 5, it is contemplated that each of surgical hair trimmer 200 and base “B” include an identification chip or tag 270a, 270b, respectively. In the present embodiment, surgical hair trimmer 200 may only be charged when docked in base “B” and when identification tags 270a, 270b register with one another.

Tags 280a, 270b may be in the form of RF identification elements, optical identification elements (e.g., bar codes, Aztec codes, etc.), and/or electrical identification elements (e.g., microchips, conductors, etc.). The RF identification elements may enable the surgical hair trimmer to be tracked, if stolen, or to sound an alarm, if removed from a designated area. In an embodiment, it is envisioned that the surgical hair trimmer may produce a signal if moved outside of a designated area, returned into a designated area or beyond a designated range (e.g., ¼ mile).

The RF identification elements may function to locate and identify the surgical hair trimmer to a particular base and/or to a particular operating room in order to potentially reduce incidents of cross-contamination and the like.

As seen in FIG. 5, blade assembly 250 may be releasably supported on handle assembly 210 in such a manner that blade housing 252 of blade assembly 250 is spaced a distance from handle housing 212 of handle assembly 210 and is substantially longitudinally movably supportable on handle assembly 210 or pivotable with respect to handle assembly 210 via hinge element 260. In this manner, during a trimming procedure, when a force “F” exerted on a bottom surface of blade assembly 250 exceeds a predetermined threshold level, blade assembly 250 is approximated toward handle assembly 210 and a spring-type cut-off switch 262 is triggered thereby stopping drive member 116. As the force “F” is reduced, and blade assembly 250 moves away from handle assembly 210, spring-type cut-off switch re-activates drive member 116. In this manner, nicking and/or cutting of the skin may be reduced.

Turning now to FIGS. 8-10, a surgical hair trimmer according to an alternate embodiment of the present disclosure is generally designated as 300. Surgical hair trimmer 300 is substantially identical to surgical hair trimmer 100 and thus will only be discussed further herein to the extent necessary to identify differences in construction and operation.

As seen in FIGS. 8-10, handle assembly 310 of surgical hair trimmer 300 includes a release mechanism 320 having at least one push button 324 or the like supported on a rear or proximal end of handle housing 312. Push button 324 is configured and adapted to operatively actuate tangs 322 to clamp onto and/or release or jettison blade assembly 350. Tangs 322 extend from a proximal end of handle housing 312 to a distal end of handle housing 312. A distal end 322b of each tang 322 is out-turned or in-turned and configured for selective engagement with corresponding engagement structure 358 formed or provided in blade assembly 350.

It is contemplated that push button 324 may be biased, by a suitable biasing member 325, such as a compression spring (see FIG. 10), to maintain push button 324 in a substantially un-actuated condition. Biasing member 325 may be disposed between a proximal shoulder 322a of tangs 322 and a shelf 327 supported in handle housing 312.

In operation, as seen in FIG. 10, as push button 324 is pressed, in the direction of arrow “A”, tangs 322 are moved in a first direction, and as push button 324 is released, tangs 322 are moved in a second direction, opposite the first direction. With blade assembly 350 connected to handle assembly 310, when push button 324 is depressed, tangs 322 are advanced distally such that distal ends 322b thereof disengage from engagement structure 358 of blade assembly 350. In so doing, blade assembly 350 is disconnected from handle assembly 310 and free to be separated therefrom.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

What is claimed is:

1. A surgical hair trimmer, comprising:
a handle assembly including:
a handle housing configured and dimensioned to house a power supply and a drive motor, wherein the drive motor includes a drive shaft extending through a distal end of the handle housing; and
a release mechanism associated with the handle housing, the release mechanism including at least one tang having a proximal end extending into the handle housing and a distal end extending out of the handle housing and being configured to engage a blade assembly; and at least one release button operatively associated with a respective tang; and
a blade assembly selectively connectable to the handle assembly, the blade assembly including:
a blade housing configured and dimensioned to house a pair of trimmer blades;
an interconnect operatively connected to one of the pair of trimmer blades to impart movement to the one trimmer blade, the interconnect being configured and adapted to selectively operatively engage the drive shaft of the drive motor when the blade assembly is connected to the handle assembly; and
an engagement structure formed in blade housing for selectively engaging the distal end of each tang of the release mechanism,
wherein when the blade assembly is connected to the handle assembly the distal end of each tang of the release mechanism of the handle assembly engages with a corresponding engagement structure of the blade housing of the blade assembly, and wherein actuation of the at least one release button of the release mechanism jettisons the blade assembly from the handle assembly.

2. The surgical hair trimmer according to claim 1, wherein a pair of release buttons are provided on side surfaces of the handle housing for operative engagement with at least a pair of tangs.

3. The surgical hair trimmer according to claim 1, wherein a single release button is provided on a rear end of the handle housing for operative engagement with at least one tang.

4. The surgical hair trimmer according to claim 1, wherein the distal end of at least one tang extends in an outward direction.

5. The surgical hair trimmer according to claim 1, further comprising at least one visual indicator supported on the handle housing.

6. The surgical hair trimmer according to claim 1, wherein a distal end of the handle housing includes a shroud extending therefrom.

7. The surgical hair trimmer according to claim 1, wherein the drive shaft of the drive motor extends through a distal wall of the handle housing, and wherein a sealing member is interposed between the drive shaft and the distal wall of the handle housing.

8. The surgical hair trimmer according to claim 1, wherein each of the release buttons is over-molded.

9. The surgical hair trimmer according to claim 1, further comprising an identification tag associated with the handle assembly for recognition with a corresponding identification tag of a charging base.

10. The surgical hair trimmer according to claim 1, for use with an anti-theft system, wherein the anti-theft system includes:
an electrical outlet connector configured and adapted for electrical connection to an electrical outlet and defining a proprietary configuration of electrical contact receptacles; and
a power cord including a power cord connector electrically connected to an end thereof, wherein the power cord connector includes electrical contacts complementing the electrical contact receptacles of the electrical outlet connector.

11. A surgical hair trimmer, comprising:
a handle assembly including a power source, a drive motor connected to the power source and having a drive shaft extending therefrom, and a release mechanism configured and adapted for single handed operation; and
a blade assembly including a pair of blades wherein at least one of the pair of blades is movable with respect to the other of the pair of blades, an interconnect configured and adapted to operatively couple the drive shaft to the movable blade when the blade assembly is connected to the handle assembly, and engagement structure configured and adapted to selectively engage the release mechanism of the handle assembly,
wherein actuation of the release mechanism, when the blade assembly is connected to the handle assembly, jettisons the blade assembly from the handle assembly.

12. The surgical hair trimmer according to claim 11, wherein the release mechanism includes at least one tang having a proximal end extending into a handle housing of the handle assembly and a distal end extending out of the handle housing, the distal end of each tang being configured to engage the blade assembly; and at least one release button operatively associated with a respective tang.

13. The surgical hair trimmer according to claim 12, wherein the engagement structure is formed in a blade housing of the blade assembly for selectively engaging the distal end of each tang of the release mechanism.

14. The surgical hair trimmer according to claim 12, wherein a pair of release buttons are provided on side surfaces of the handle housing for operative engagement with at least a pair of tangs.

15. The surgical hair trimmer according to claim 12, wherein a single release button is provided on a rear end of the handle housing for operative engagement with at least one tang.

16. The surgical hair trimmer according to claim 11, wherein the handle assembly further includes at least one visual indicator.

17. The surgical hair trimmer according to claim 11, wherein a distal end of a handle housing of the handle assembly includes a shroud extending therefrom.

18. The surgical hair trimmer according to claim 11, wherein the drive shaft of the drive motor extends through a distal wall of a handle housing of the housing assembly, and wherein a sealing member is interposed between the drive shaft and the distal wall of the handle housing.

19. The surgical hair trimmer according to claim 12, wherein each release button is over-molded.

20. The surgical hair trimmer according to claim 11, further comprising an identification tag associated with the handle assembly for recognition with a corresponding identification tag of a charging base.

21. The surgical hair trimmer according to claim 11, for use with an anti-theft system, wherein the anti-theft system includes:
an electrical outlet connector configured and adapted for electrical connection to an electrical outlet and defining a proprietary configuration of electrical contact receptacles; and
a power cord including a power cord connector electrically connected to an end thereof, wherein the power cord connector includes electrical contacts complementing the electrical contact receptacles of the electrical outlet connector.
